(19) United States
(12) Patent Application Publication
(10) Pub. No.: US 2007/0282233 A1
(43) Pub. Date: Dec. 6, 2007

(54) COMPRESSION APPARATUS

(75) Inventors: Ann P. Meyer, Shrewsbury, MA (US); Kristin Watson, North Attleboro, MA (US); Heather L. Gillis, Sharon, MA (US); Mark A. Vess, Hanson, MA (US); Malcolm G. Bock, Medfield, MA (US)

Correspondence Address: TYCO HEALTHCARE - EDWARD S. JARMOLOWICZ, 15 HAMPSHIRE STREET, MANSFIELD, MA, 02048, US

(73) Assignee: TYCO HEALTHCARE GROUP LP, Mansfield, MA (US)

(21) Appl. No.: 11/761,212

(22) Filed: Jun. 11, 2007

Related U.S. Application Data
(63) Continuation-in-part of application No. 11/299,568, filed on Dec. 12, 2005.
(63) Continuation-in-part of application No. 11/760,321, filed on Jun. 8, 2007.

(51) Int. Cl. A61F 5/00; B32B 3/00

(52) U.S. Cl. 602/23; 428/71; 602/10; 602/27

(57) ABSTRACT

A compression apparatus is a foot cuff having an inner and outer layer with an inflatable member disposed therein. The inflatable member is attached to the inner or outer layer of the foot cuff by a variety of methods that leave it free to move with respect to the unattached layer.

This application is a continuation-in-part of U.S. Ser. No. 11/299,568, filed Dec. 12, 2005, the entire contents of which are incorporated herein by reference, and is a continuation-in-part of U.S. Ser. No. 11/760,321, filed Jun. 8, 2007, the entire contents of which are incorporated herein by reference.

### BACKGROUND

The present disclosure relates generally to compression apparatus. In particular, the present disclosure relates to a compression apparatus configured for applying compressive forces to a portion of a patient's anatomy.

Compression devices for applying compressive forces to a selected area of a person's anatomy are generally employed to improve blood flow in the selected area. Compression devices that provide intermittent pulses of a compressed fluid (i.e. air) to inflate at least one inflatable chamber in a cuff are particularly useful. This cyclic application of pressure provides a non-invasive method of prophylaxis to reduce the incidence of deep vein thrombosis (DVT), and the like. These compression devices find particular use during surgery on patients with high-risk conditions such as obesity, advanced age, malignancy, or prior thromboembolism. Patients who develop this condition often have swelling (edema) and tissue breakdown (venous stasis ulcer) in the lower leg. When a DVT occurs, the valves that are located within the veins of the leg can be damaged, which in turn can cause stasis and high pressure in the veins of the lower leg.

Generally, these compression devices are fluidly coupled to a source of pressurized fluid by one or more air tubes. Additionally, each compression device includes a flexible shell having one or more inflatable members disposed therein. The compression device is placed around the patient's foot or other selected portion whereupon a pressurized fluid is delivered into the inflatable member creating pressure at the part or parts of the body in contact with the inflatable member.

Compression cuffs adapted for use with a patient's foot may be combined with one or more additional compression cuffs that are disposed on portions of a patient's leg for improving the treatment regimen. In general, each of the additional compression cuffs includes a plurality of separate inflatable chambers that are progressively arranged along a longitudinal axis of the cuff from a lower portion to an upper portion of the limb. A pressure source, e.g. a controller, is provided for intermittently forming a pressure pulse within these inflatable chambers from a source of pressurized fluid during periodic compression cycles. The compression cuffs provide a pressure gradient along the patient's limbs during these compression cycles which progressively decreases from the lower portion to the upper portion of the limb (e.g. from the ankle to the thigh).

Compression cuffs that are adapted for use with a patient's foot generally include a heel strap with a tab portion that is adapted to fit around a portion of the patient's heel. This arrangement allows the compression cuff to be releasably attached to the patient's foot.

Examples of compression cuffs are disclosed in U.S. Pat. Nos. 4,013,069 and 4,030,488 to Hasty, U.S. Pat. Nos. 4,029,087 and 5,795,312 to Dye, and U.S. Pat. No. 5,626,556 to Tobler et al., all of which are currently owned by Tyco Healthcare Group LP and are incorporated by reference herein in their entireties. Other examples of compression cuffs are disclosed in U.S. Pat. Nos. 4,696,289 to Gardner et al. and 5,989,204 to Lina. An example of compression treatment method is disclosed in U.S. Pat. No. 6,231,532 to Watson et al., which is currently owned by Tyco Healthcare Group LP, the contents of which are hereby incorporated by reference herein in their entirety.

Some prior art devices are bulky and may irritate portions of the limb undergoing treatment which may increase patient discomfort and may increase the possibility that the patient may not complete the treatment regimen.

### SUMMARY

In one aspect, a compression apparatus includes a first layer and a second layer. The first layer and second layer define a space between them. A single inflatable member is disposed in the space. The single inflatable member is freely movable in relation to at least one of the first and second layers when it is disposed on the body part of a patient for providing DVT prophylaxis therapy to a limb. An outer surface of the inflatable member is secured to an inner surface of the first or second layer by an adhesive layer. The adhesive layer is selected from the group consisting of at least one strip of adhesive tape, an adhesive fluid, a gel and combinations thereof.

In another aspect, a compression apparatus includes a first layer and a second layer. The first layer and second layer define a space between them. A single inflatable member is disposed in the space. The single inflatable member is freely movable in relation to at least one of the first and second layers when it is disposed on the body part of a patient for providing DVT prophylaxis therapy to a limb. A fastener secures an outer surface of the inflatable member to an inner surface of the first or second layer. The fastener is selected from the group consisting of a button, a snap, a hook and eye fastener, a hook and loop fastener, a staple, a zip fastener, a magnetic fastener, a rivet and combinations thereof.

In another aspect, a compression apparatus includes a first layer and a second layer. The first layer and second layer define a space between them. A single inflatable member is disposed in the space. The single inflatable member is freely movable in relation to at least one of the first and second layers when it is disposed on the body part of a patient for providing DVT prophylaxis therapy to a limb. Stitching joins an outer surface of the inflatable member to an inner surface of the first or second layer.

In a further aspect, a compression apparatus includes a first layer and a second layer. The first layer and second layer define a space between them. A single inflatable member is disposed in the space. The single inflatable member is freely movable in relation to at least one of the first and second layers when it is disposed on the body part of a patient for providing DVT prophylaxis therapy to a limb. An outer surface of the inflatable member is secured to an inner surface of the first or second layer by fusion of the outer surface of the inflatable member with the inner surface of the first or second layer.

In still a further aspect, a compression apparatus includes a first layer and a second layer. The first layer and second layer define a space between them. A single inflatable member is disposed in the space. The single inflatable member is freely movable in relation to at least one of the first and second layers when it is disposed on the body part of a patient for providing DVT prophylaxis therapy to a limb. A crimped element is located between the inflatable member and the first or second layer. The crimped element has folds capturing portions of the inflatable member and first or second layer for securing an outer surface of the inflatable member to an inner surface of the first or second layer.

In another aspect, a compression apparatus includes a first layer and a second layer. The first layer and second layer define a space between them. A single inflatable member is disposed in the space. The single inflatable member is freely movable in relation to at least one of the first and second layers when it is disposed on the body part of a patient for providing DVT prophylaxis therapy to a limb. A material of an outer surface of the inflatable member and a material of an inner surface of the first or second layer are selected to produce an electrostatic connection of the outer surface of the inflatable member with the inner surface of the first or second layer.

In yet another aspect a compression apparatus includes a first layer and a second layer. The first layer and second layer define a space between them. A single inflatable member is disposed in the space. The single inflatable member has only one chamber or one bladder for holding fluid during a compression treatment cycle. In one embodiment, a bladder is defined as two sheets of an impervious material, such as PVC, welded to form a single area with an opening for introducing and exhausting fluid during a compression treatment cycle. The single inflatable member is freely movable in relation to at least one of the first and second layers when it is disposed on the body part of a patient for providing DVT prophylaxis therapy to a limb. Straps are attached to the inner surface of the first or second layer. The straps receive portions of the inflatable member for securing an outer surface of the inflatable member to an inner surface of the first or second layer.

Other features will be in part apparent and in part pointed out hereinafter. Various refinements exist of the features noted in relation to the above-mentioned aspects of the present invention. Further features may also be incorporated in the above-mentioned aspects of the present invention as well. These refinements and additional features may exist individually or in any combination. For instance, various features discussed below in relation to any of the illustrated embodiments of the present invention may be incorporated into any of the above-described aspects.

### BRIEF DESCRIPTION OF THE DRAWINGS

The features of the presently disclosed compression apparatus will become more readily apparent by referring to the following detailed description of embodiments, which are described hereinbelow with reference to the drawings, wherein:

FIG. 1 is an exploded perspective view, of a first embodiment of the compression apparatus in accordance with the present disclosure;

FIG. 2 is a top plan view of the compression apparatus illustrated in FIG. 1;

FIG. 3 is a top plan view of the compression apparatus of FIG. 1, illustrating the outline of a foot of a patient disposed thereon;

FIG. 4 is a bottom plan view of the compression apparatus of FIG. 1; FIGS. 5-6 are bottom plan views of the compression apparatus of FIG. 1 indicating inflatable member attachment points;

FIG. 7 is a fragmentary cross-section view of the outer layer and inflatable member of the compression apparatus of FIG. 1 illustrating attachment by use of an adhesive layer;

FIG. 8 is a fragmentary cross-section view of the outer layer and inflatable member of the compression apparatus of FIG. 1 illustrating attachment by use of magnetic strips;

FIG. 9 is a fragmentary cross-section view of the outer layer and inflatable member of the compression apparatus of FIG. 1 illustrating attachment by fusion;

FIG. 10 is a fragmentary cross-section view of the outer layer and inflatable member of the compression apparatus of FIG. 1 illustrating attachment by use of buttons;

FIG. 11 is a fragmentary cross-section view of the outer layer and inflatable member of the compression apparatus of FIG. 1 illustrating attachment by use of snaps;

FIG. 12 is a fragmentary cross-section view of the outer layer and inflatable member of the compression apparatus of FIG. 1 illustrating attachment by use of hook and eye fasteners;

FIG. 13 is a fragmentary cross-section view of the outer layer and inflatable member of the compression apparatus of FIG. 1 illustrating attachment by hook and loop fasteners;

FIG. 14 is a fragmentary cross-section view of the outer layer and inflatable member of the compression apparatus of FIG. 1 illustrating attachment by staples;

FIG. 15 is a fragmentary cross-section view of the outer layer and inflatable member of the compression apparatus of FIG. 1 illustrating attachment by rivets;

FIG. 16 is a fragmentary cross-section view of the outer layer and inflatable member of the compression apparatus of FIG. 1 illustrating attachment by stitching;

FIG. 17 is a fragmentary cross-section view of the outer layer and inflatable member of the compression apparatus of FIG. 1 illustrating attachment by electrostatic connection.

FIG. 18 is a fragmentary cross-section view of the outer layer and inflatable member of the compression apparatus of FIG. 1 illustrating attachment by use of zip fasteners;

FIG. 19 is a perspective of the compression apparatus of FIG. 1 with an inner layer removed therefrom, illustrating attachment of the inflatable member and outer layer by straps;

FIG. 20 is a fragmentary cross-section view of the outer layer and inflatable member of the compression apparatus of FIG. 1 illustrating attachment by use of a crimping element;

FIGS. 21-24 are top plan views of further alternative embodiments of a compression apparatus, in accordance with the present disclosure;

FIG. 25 is a top plan view of the compression apparatus of FIG. 1 disposed about a foot of a patient;

FIG. 26 is a side plan view of the compression apparatus of FIG. 1 disposed about a foot of a patient;

FIG. 27 is a perspective view of another embodiment of the compression apparatus, in accordance with the present disclosure; and

FIG. 28 is a perspective view illustrating the compression apparatus of FIG. 27 wrapped around a leg of a patient.

### DETAILED DESCRIPTION

With reference to the drawing figures, in which like references numerals identify identical or corresponding elements, various embodiments of the presently disclosed compression apparatus will now be described in detail.

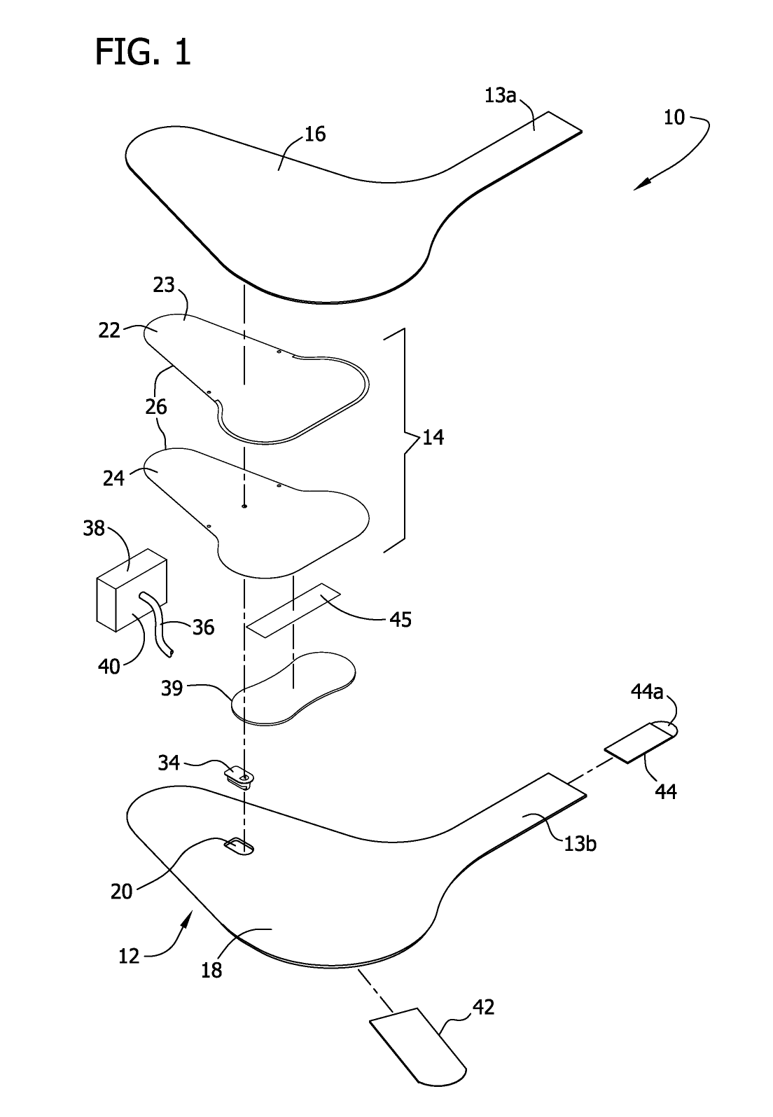

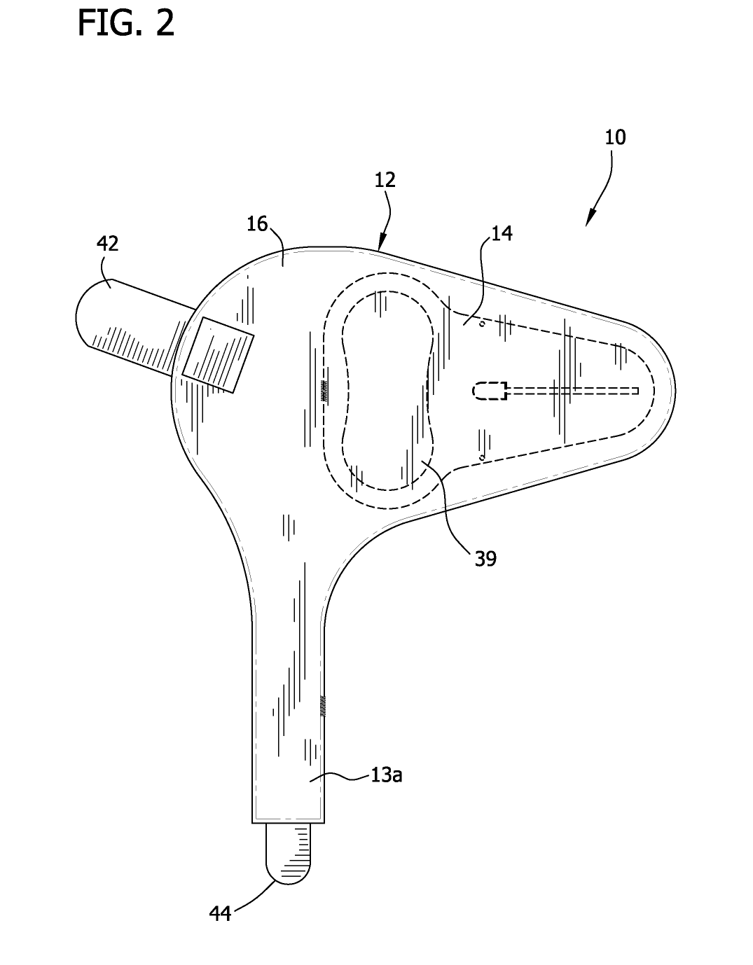

With initial reference to FIGS. 1-4, a first embodiment of a compression apparatus in accordance with the present disclosure is illustrated and is designated generally as compression apparatus 10. Compression apparatus 10 is adapted for use in a system for applying compressive pressure to a portion of a body, such as, for example, a foot of a person. Compression apparatus 10 generally includes a foot cuff 12 configured for disposal about a foot and an inflatable member 14 disposed within foot cuff 12 and being freely movable or repositionable in relation to foot cuff 12. Moreover, foot cuff 12 is configured and dimensioned for disposing about the right or left foot of the subject.

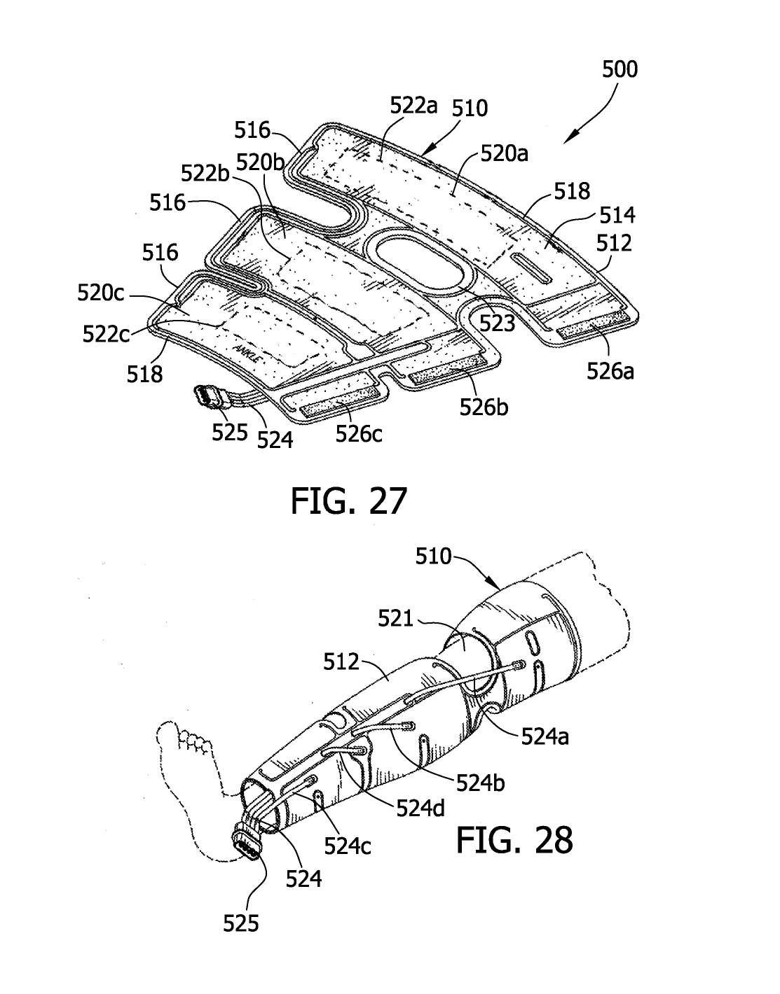

Foot cuff 12 includes an inner or contact layer 16 and an outer layer 18 fixedly joined at seams adjacent corresponding perimeters thereof and defining a space for receiving inflatable member 14 therein. Inflatable member 14 forms a single chamber with an opening for allowing fluid to be introduced or exhausted during a compression cycle. In this embodiment, member 14 is only a single bladder for providing compression. Referring to FIG. 27, inflatable members 522a, 522b and 522c have only one single bladder. The inlet 34 provides for air or fluid to be introduced during the start of a compression cycle and to be exhausted to end the compression cycle. The forming of the single bladder inflatable member is described fixedly joined at its seams. The single inflatable member 14 has only one chamber or one bladder for holding fluid during a compression treatment cycle. A bladder is defined as two sheets of an impervious material, such as PVC, welded to form a single area with an opening for introducing and exhausting fluid during a compression treatment cycle. Contact layer 16 and outer layer 18 may be joined by radio frequency (RF) welding, sewing, adhesives, etc. Contact layer 16 and outer layer 18 include strap portions 13a and 13b respectively. Strap portions 13a, 13b have a longitudinally projecting configuration for wrapping about a portion of the foot adjacent to the ankle. Contact layer 16 is adapted for contacting the bottom portion of the foot and providing a fabric on fabric slide with inflatable member 14, in accordance with the present disclosure. Strap portions 13a, 13b can be sewn, RF welded, or sonic welded. However in the illustrated embodiments, the strap portions 13a,13b are formed as one piece with the contact layer 16 and outer layer 18, respectively. Contact layer 16 is fabricated from a chemically treated material, with wicking ability, for wicking away moisture from the skin. In one embodiment, contact layer 16 includes a mesh-like fabric capable of wicking moisture away from the patient's skin. Furthermore, the contact layer 16 can be faced with a soft material toward the treatment surface of the patient. The material can be a thin layer of open celled porous foam, napped cloth, or a layer of vapor permeable cloth permeable.

Outer layer 18 includes an opening 20 for permitting a pressurized fluid inlet passage therethrough, in a manner described herein below. Outer layer 18 is configured for providing the attachment surface for a hook and loop feature of compression apparatus 10, as will be described in detail hereinbelow. Moreover, outer layer 18 provides a soft material for cushioning effect against the top portion of the feet and may be fabricated from similar materials as contact layer 16 and in similar dimensions therewith for corresponding geometry. Alternatively, outer layer 18 may be fabricated from a laminated material, such as, for example, sontara fabric, open cell urethane foam, or loop fabric.

With particular reference to FIG. 1, inflatable member 14 is disposed within the space defined by contact layer 16 and outer layer 18 and is configured for moving independently relative to foot cuff 12. Inflatable member 14 is configured for positioning against the bottom portion of the foot and the ankle portion. Inflatable member 14 is adapted for receiving and retaining a pressurized fluid (e.g. air) for exerting compressive pressure to the foot during successive pressure applying cycles. Inflatable member 14 includes upper and lower inflatable layers 22, 24 overlaid to form an inflatable portion. Upper and lower layers 22, 24 are fixedly joined via sealing lines 26 along their perimeters to define the inflatable portion. Sealing lines 26 may be formed by radio frequency (RF) welding. Alternatively, sealing lines 26 may be sewn, formed by adhesive, heat sealing, etc.

A first surface 23 of upper inflatable layer 22 is positioned just below contact layer 16 for providing the largest compression effect on the foot. Upper inflatable layer 22 includes material for wicking away moisture from the bottom of the feet. In one embodiment, upper inflatable layer 22 includes a two-part laminated material that is formed from a chemically treated wicking fabric or non-woven material combined with a PVC or urethane sheet thereby allowing layer 22 to move with respect to contact layer 16. In another embodiment, upper inflatable layer 22 is a PVC or urethane sheet allowing layer 22 to move with respect to contact layer 16.

Lower inflatable layer 24 includes a single material such as a polyvinyl chloride (PVC). It is envisioned that the material used to fabricate the inflatable member 14 may include at least two different thicknesses for providing directional inflation of inflatable member 14. Thus, inflation of inflatable member 14 yields different shapes as determined by the thickness of inflatable member 14.

The inflatable member 14 may be attached to the contact layer 16 or the outer layer 18. It is preferred that the inflatable member 14 be attached to the outer layer 18 as this permits relative movement between contact layer 16 and the inflatable member 14. As a result, the skin does not become irritated or chaffed at locations where the contact layer 16 contacts the skin during the cyclical application of compression.

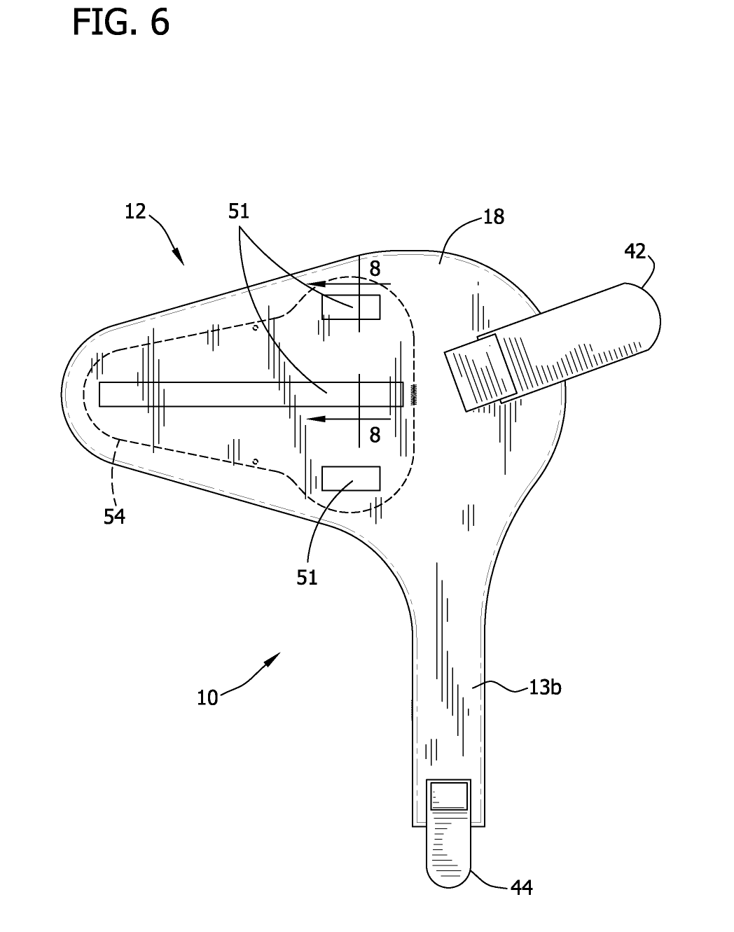

The inflatable member 14 may be attached to the outer layer 18 by a variety of methods. Attachment of the inflatable member to the outer layer 18 may occur (for example) at all locations of contact between the inflatable member 14, at discrete locations (FIG. 5; indicated by “X”), along attachment strips 51 (FIG. 6) or along the inflatable member perimeter 54. Various methods of attachment illustrated in FIGS. 7-20 will now be described. While attachment of the inflatable member 14 to the outer layer 18 is illustrated, it should be understood that the illustrated fastening methods are also suitable for attachment of the inflatable member 14 to the contact layer 16.

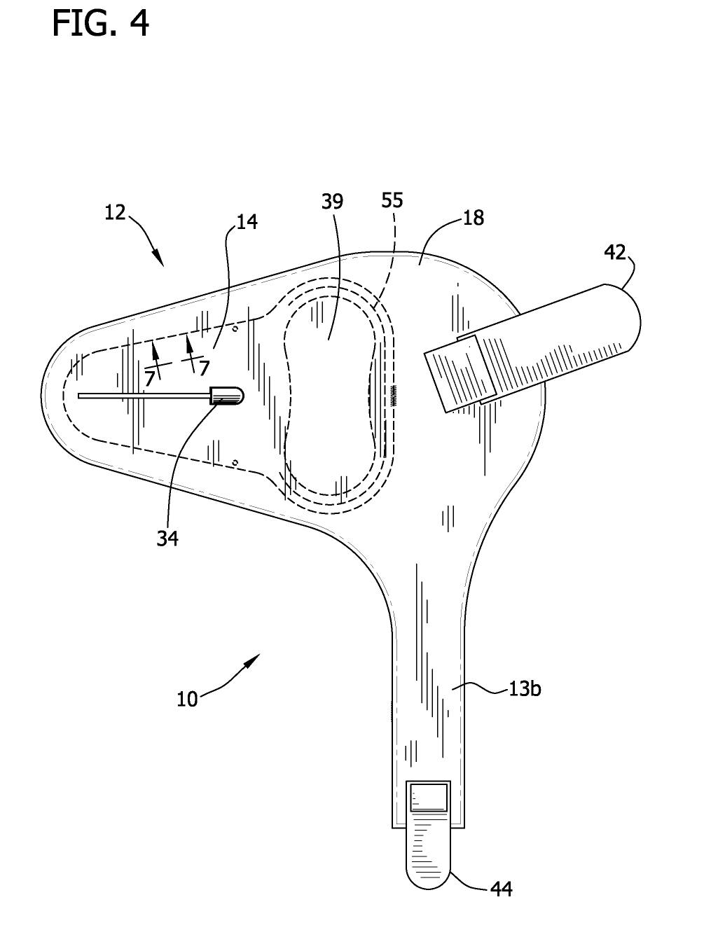

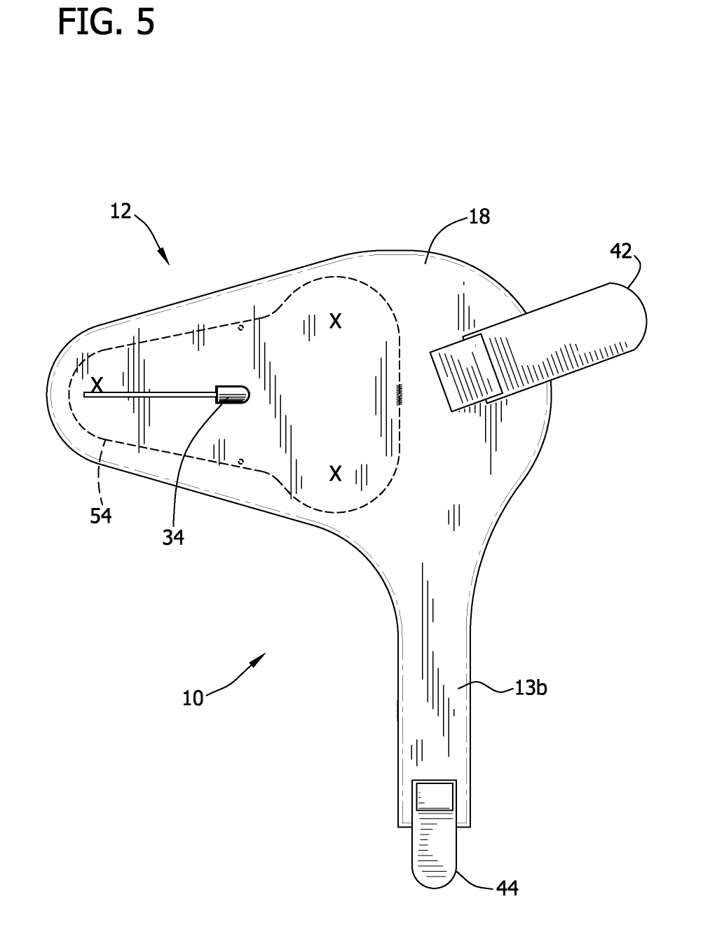

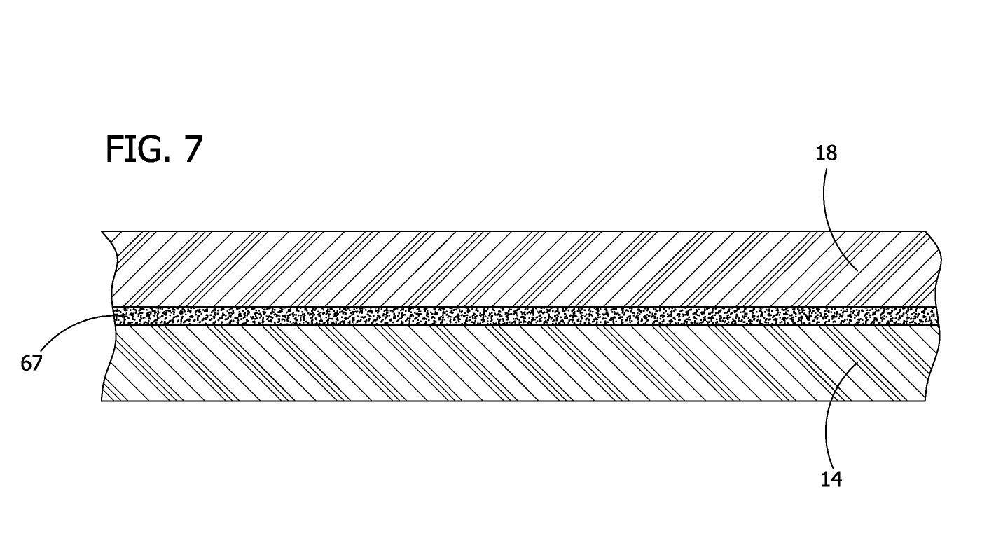

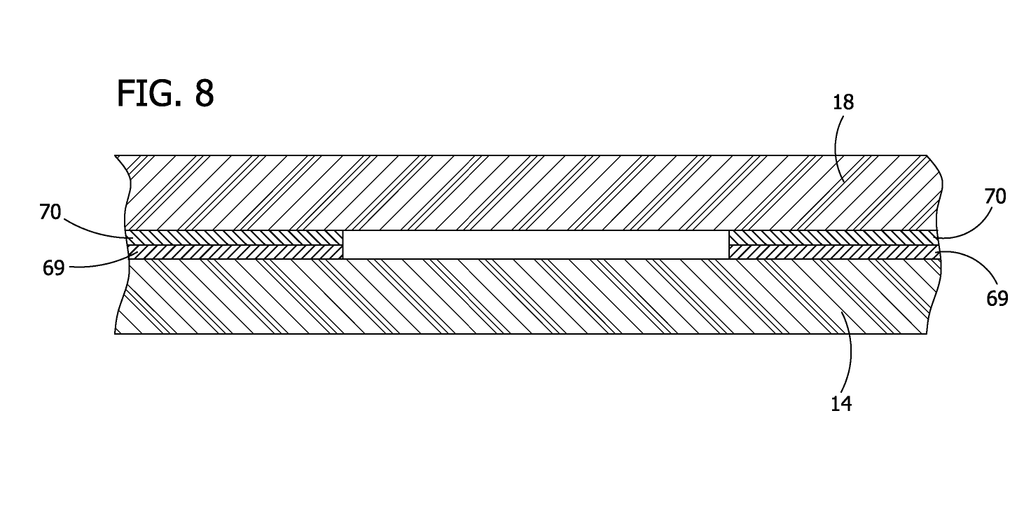

Referring to FIG. 7, a fragmentary cross-section of the inflatable member 14 and outer layer 18 is taken generally as indicated by line 7-7 of FIG. 4. All other components of the foot cuff 12 are removed for clarity of illustration. The inflatable member 14 is attached to the outer layer 18 by a layer of adhesive 67. This layer of adhesive 67 can be coextensive with the outer surface of the inflatable member 14 or formed by discrete adhesive “strips” 51 or “tape” as shown in FIG. 6. The adhesive layer 67 may be constructed of, for example, adhesive tape, adhesive fluids, gels or combinations thereof. Suitable adhesive fluids include, for example, glues, thermoplastic adhesives, thermosetting adhesives, rubber-resin blendsultraviolet curable adhesives and mixtures thereof. Suitable UV curable adhesives include, for example, acrylated urethanes, urethane oligomers and mixtures thereof. Suitable gels include, for example, silicone gels, hydrocolloidal gels, cyanoacrylate gels, thixotropic gels and mixtures thereof.Referring to FIG. 8, the inflatable member 14 is attached to the outer layer 18 by use of magnetic strips. FIG. 8 is similar to FIG. 7 but may be taken as indicated by line 8-8 of FIG. 6. Two magnetic strips 69 are attached to the inflatable member 14 and two magnetic strips 70 are attached to the outer layer 18. The magnetic strips can be attached to the inflatable member 14 and outer layer 18 in any suitable manner, for example, by adhesives. The magnetic strips should be configured so that the magnetic poles of each magnetic strip are aligned with the opposite magnetic pole of its mating magnetic strip. The arrangement of the strip or strips can be other than that shown within the scope of the present invention.

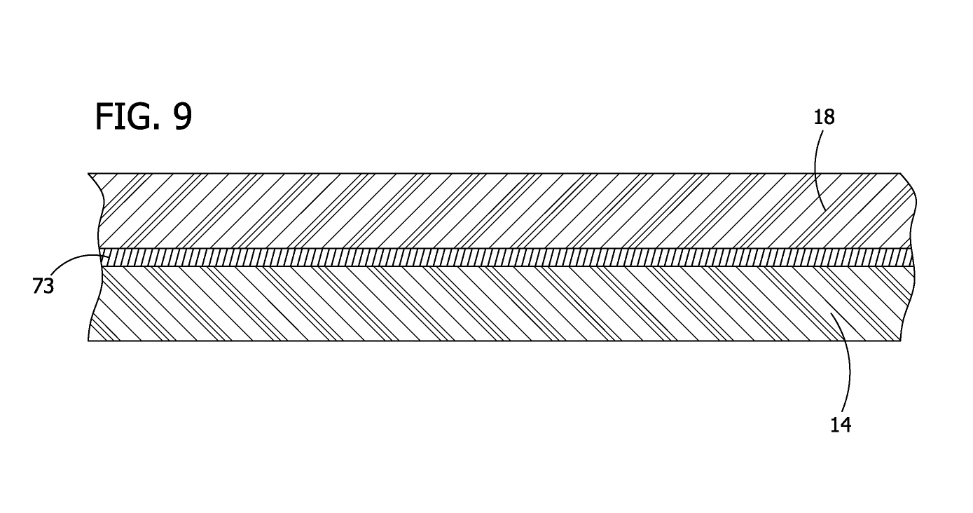

Referring to FIG. 9, a cross section similar to FIG. 7, the inflatable member 14 is secured to the outer layer 18 by fusing the inflatable member 14 to the outer layer 18. This fusion creates an “intermingled” layer 73 which comprises the fused portions of both layers. However, the material of only one of the inflatable member and contact layer may form the intermingled layer 73 (i.e., only one of the layers may melt). The inflatable member 14 may be fused to the outer layer 18 by, for example, heat lamination, radiofrequency welding or by solvent bonding.

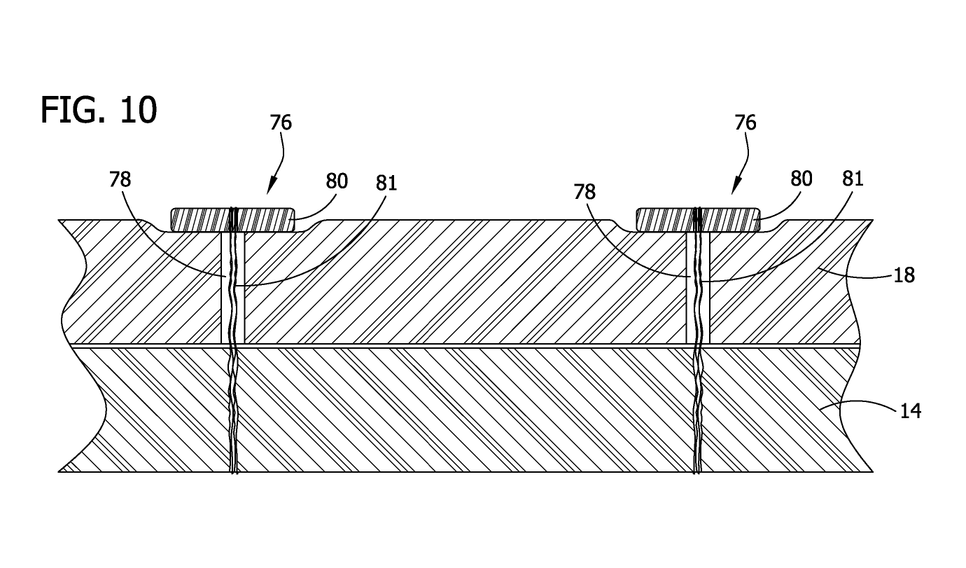

Referring to FIG. 10, the inflatable member 14 is attached to the outer layer 18 by two buttons 76. Each button 76 comprises a catch 80 that is sewn to the inflatable member 14 by threads 81. The inflatable member 14 is secured by fitting each catch 80 through a slit 78 formed in the outer layer 18.

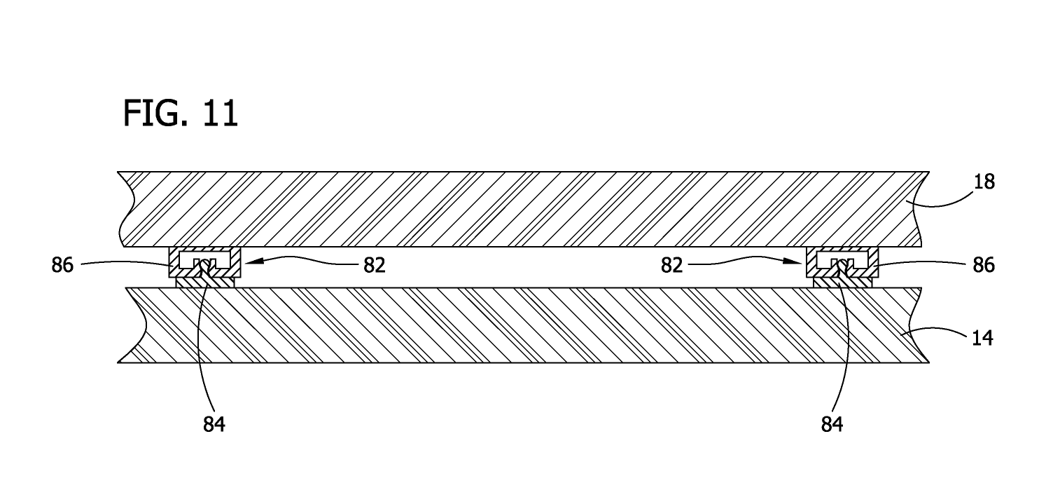

Referring to FIG. 11, the inflatable member 14 is secured to the outer layer 18 by two snaps 82. Each snap 82 comprises a first snap element 84 attached to the inflatable member 14 and a second snap element 86 attached to the outer layer 18. The first snap element 84 and second snap element 86 are adapted for releasable, snap-in connection. The first snap element 84 and second snap element 86 are attached to the inflatable member 14 and outer layer 18, respectively, by any suitable means including, for example, adhesion or by press-fitting the respective layer between metal flanges attached to the snap member.

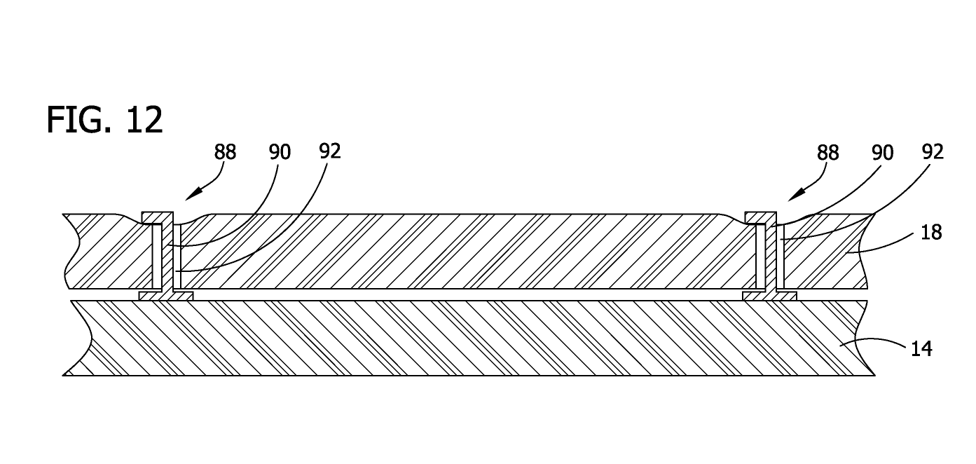

Referring now to FIG. 12, the inflatable member 14 is attached to the outer layer 18 by a hook and eye fastener 88. The hook and eye fasteners 88 include a hook 90 attached to the inflatable member 14 which extends through an opening or “eye” 92 in the outer layer 18. The hook 88 may be attached to the inflatable member by any suitable means including, for example stitching.

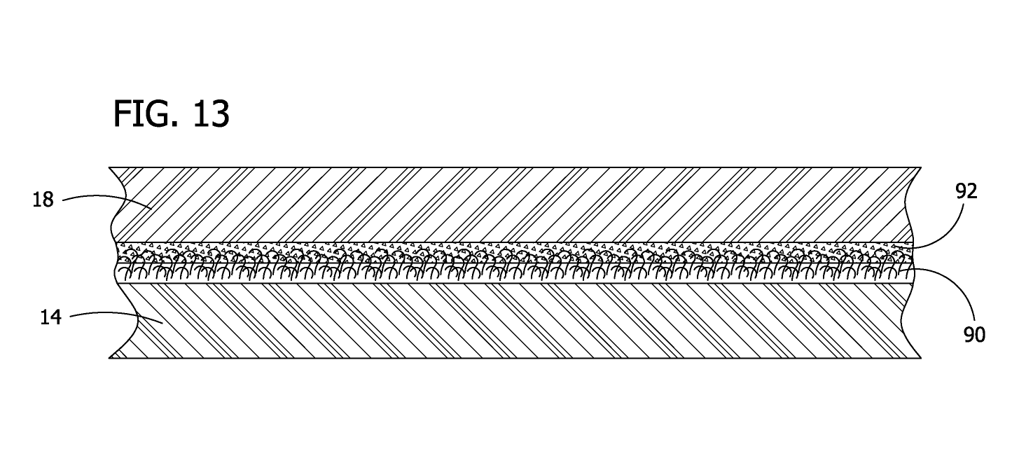

Referring now to FIG. 13, the inflatable member 14 is attached to the outer layer 18 by hook elements 90 and loop elements 92. While the hook elements 90 are shown attached to the inflatable member 14 and the loop elements 92 are shown attached to the outer layer 18, the reverse arrangement is within the scope of the invention.

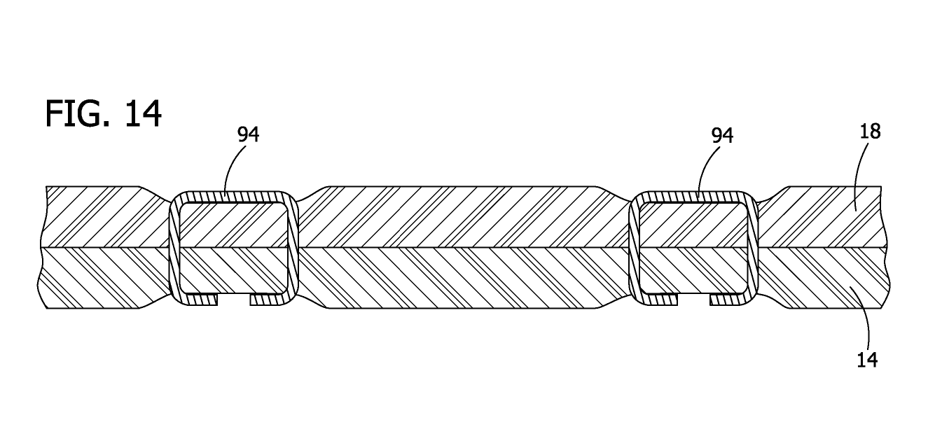

Referring now to FIG. 14, the inflatable member 14 is attached to the outer layer 18 by staples 94.

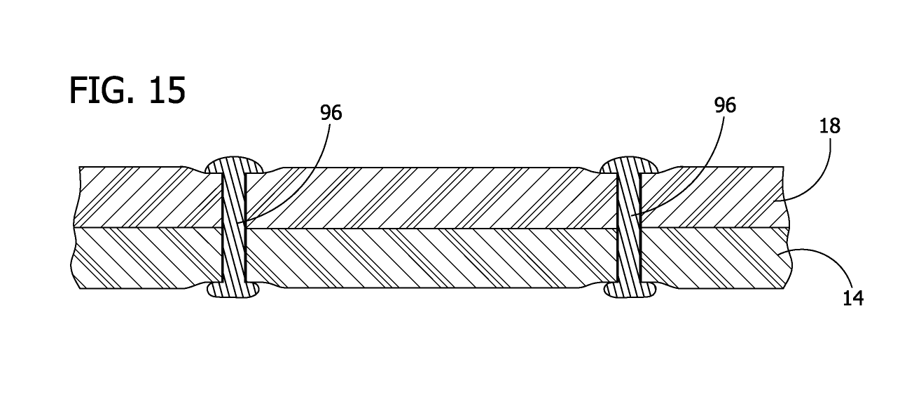

Referring now to FIG. 15, the inflatable member 14 is attached to the outer layer 18 by rivets 96.

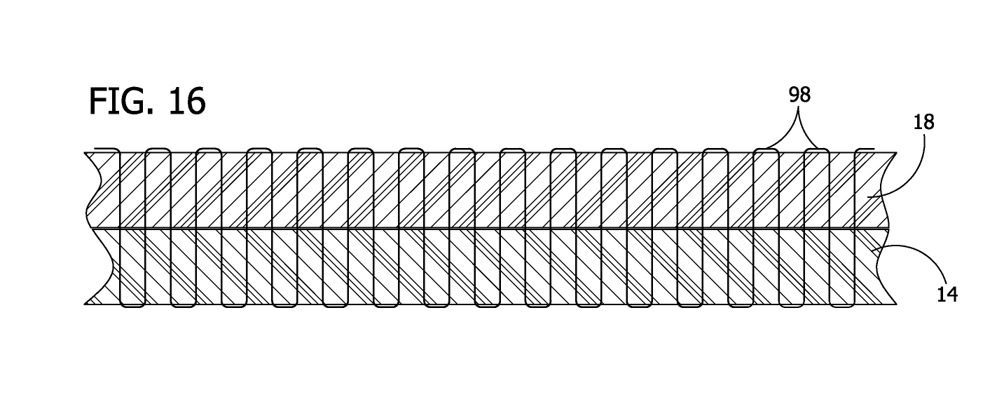

Referring now to FIG. 16, the inflatable member 14 is attached to the outer layer 18 by stitching 98 which joins an outer surface of the inflatable member to the inner surface of the outer layer 18. The stitching 98 can be located along the periphery 54 (FIG. 6), away from the periphery in any suitable pattern or at discrete points (FIG. 5).

Referring now to FIG. 17, the inflatable member 14 is attached to the outer layer 18 by electrostatic connection. The material of the outer surface of the inflatable member 14 and the inner surface of the outer layer 18 are selected such that they contain opposite charges which causes an electrostatic attraction. Suitable materials which cause a positive charge, i.e., which tend to donate electrons, include, for example, leather, nylon and wool. Suitable materials which cause a negative charge, i.e., which readily accept electrons, include, for example polyurethane, polyethylene, polypropylene and polyvinyl chloride.

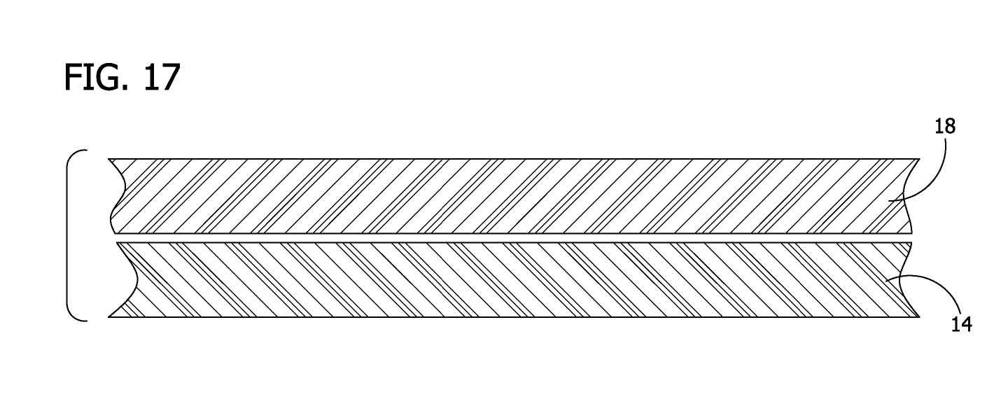

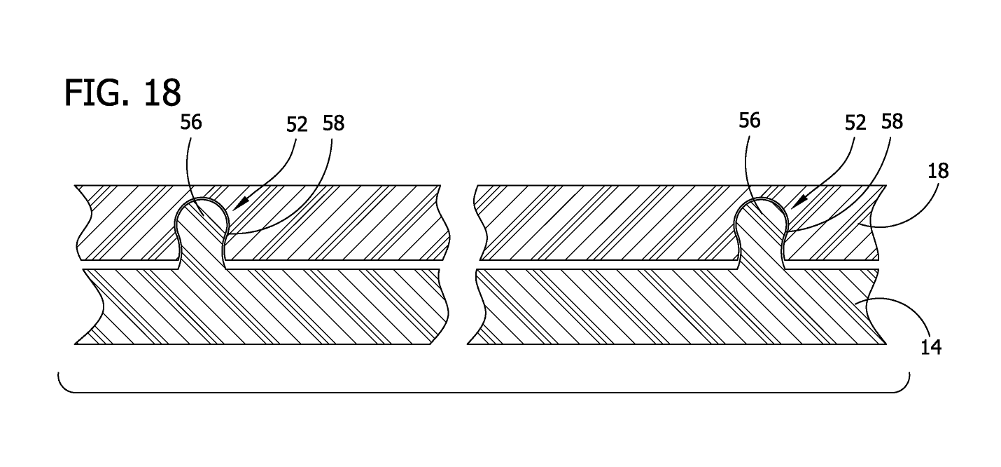

Referring to FIG. 18, the inflatable member 14 is attached to the outer layer 18 by zip fasteners 52 (e.g., like a ZIPLOC fastener). The zip fasteners 52 include a groove element 58 formed within the outer layer 18 and a tongue 56 which extends from the inflatable member 14. At least a portion of the tongue 56 is larger than the width of the groove element 58. The outer walls of the groove element 58 are resiliently deformable such that the tongue 56 can be releasably captured within the groove upon mating of the tongue and groove element. The zip fastener 52 may extend around the perimeter or over shorter segments at or away from the perimeter of the inflatable member 14. The tongue 56 may be integral with the inflatable member 14 or may attached thereto by, for example, adhesive or RF welding.

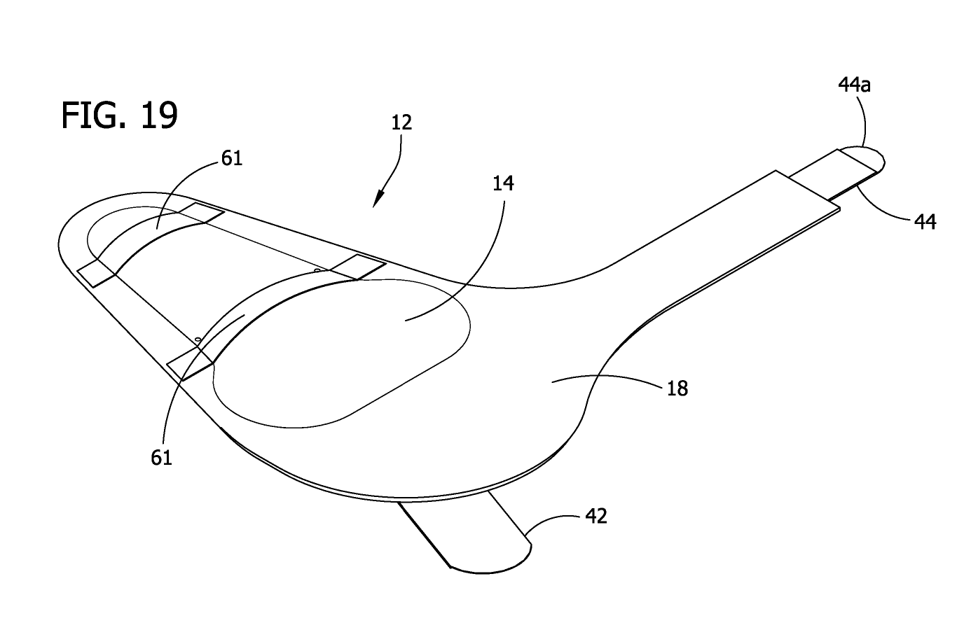

Referring to FIG. 19, the foot cuff 12 is shown with the contact layer 16 removed. The inflatable member 14 is secured to the outer layer 18 by two straps 61 which receive portions of the inflatable member. The number and/or arrangement of the straps may be other than shown. The straps 61 are attached to the inner surface of the outer layer 18 by any suitable method including stitching, RF welding or heat lamination.

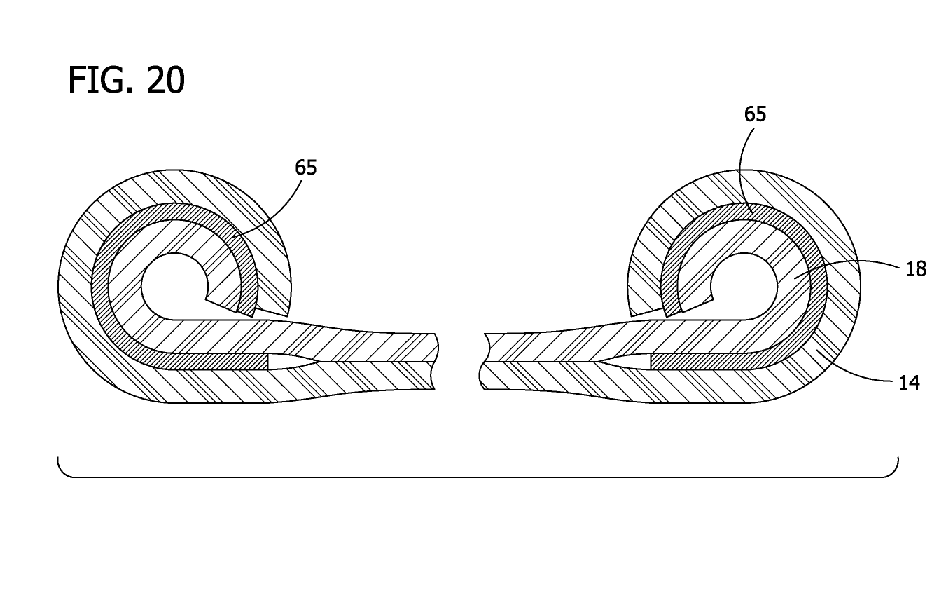

Referring to FIG. 20, the inflatable member 14 is secured to the outer layer 18 by use of a crimping element 65. The crimping element 65 is either rounded or folded such that it captures portions of the inflatable member 14 and outer layer 18 to secure the outer surface of the inflatable member and inner surface of the outer layer together. The crimping element 65 may be composed of any resilient material capable of retaining its shape after being rolled or crimped (broadly, “folded”) upon itself. Suitable materials include, for example, thin sheets of metal or resilient plastic. The crimpled member 65 may extend continuously along the perimeter of the inflatable member 14 or may extend along discrete tab portions (not shown) along the perimeter. Any discrete tab portions are typically defined by two slits formed through the inflatable member 14 and outer layer 18 and formed perpendicular to the cuff perimeter.

Referring again to FIGS. 1-4, the cuff 12 includes a sole member 39. The sole member 39 is generally shaped according to contours of the sole of the foot. The sole member 39 is made of a rigid material, for example, metal, polymers, ceramics or composite-type materials. The rigidity of the sole member 39 helps to direct the compression supplied by the inflatable member 14 toward the foot.

The sole member 39 may be located between the upper inflatable layer 22 of the inflatable bladder 14 and the contact layer 16, however, it is preferred that the sole member 39 be located between the lower inflatable layer 24 and the outer layer 18 as illustrated in FIG. 1. This arrangement allows compression to be more directly applied to the foot.

The sole member 39 is attached to the inflatable member 14 by a strip of adhesive tape 45. Alternatively or in addition, the position of the sole member 39 relative to the contact layer 16 and outer layer 18 may be restricted by stitching 55 (FIG. 4) which partially extends around the perimeter of the sole member. The stitching 55 also connects a wider portion of the inflatable member 14 to the outer layer 18. A narrower portion or “tongue” of the inflatable member 14 could be free floating with respect to both the contact layer 16 and the outer layer 18. [ESJ—is→ could be provide a claim is not made indefinite]. Other methods of attaching and retaining the sole member are contemplated within the scope of the present invention, including, for example, stitching, RF welding, heat lamination and solvent bonding.

In embodiments where the sole member 39 is not included in the cuff 12, the inflatable member 14 can be fastened directed to the outer layer without the sole member being interposed therebetween. In embodiments where the sole member 39 is included in the cuff 12, the sole member may include one or more of the fastening/attachment elements describe above for attaching the sole member to the outer layer 18.

Referring now to FIGS. 1 and 4, a plurality of hook fasteners 42, 44 are provided for attaching compression apparatus 10 to a foot F, and are positioned on outer layer 18 of foot cuff 12. Hook 44 is mounted to strap portion 13b of outer layer 18 of foot cuff 12 while hook 42 is mounted on a surface of outer layer 18. In use, when strap portions 13a,13bare wrapped about foot F, hook element 44 engages outer layer 18 to facilitate mounting of foot cuff 12 to foot F. In addition, inflatable strap portion 21 of inflatable member 14 is disposed about foot F for compression therapy. An identification tab (not shown) may also be included for providing information such as the model number and manufacturer name. Hook fasteners 42, 44 may have tabs (one shown, 44a) without fastening material thereon to provide convenient gripping locations on the hook fasteners to thereby allow the practitioner to easily remove hooks 42, 44 from the surface of outer layer 18.

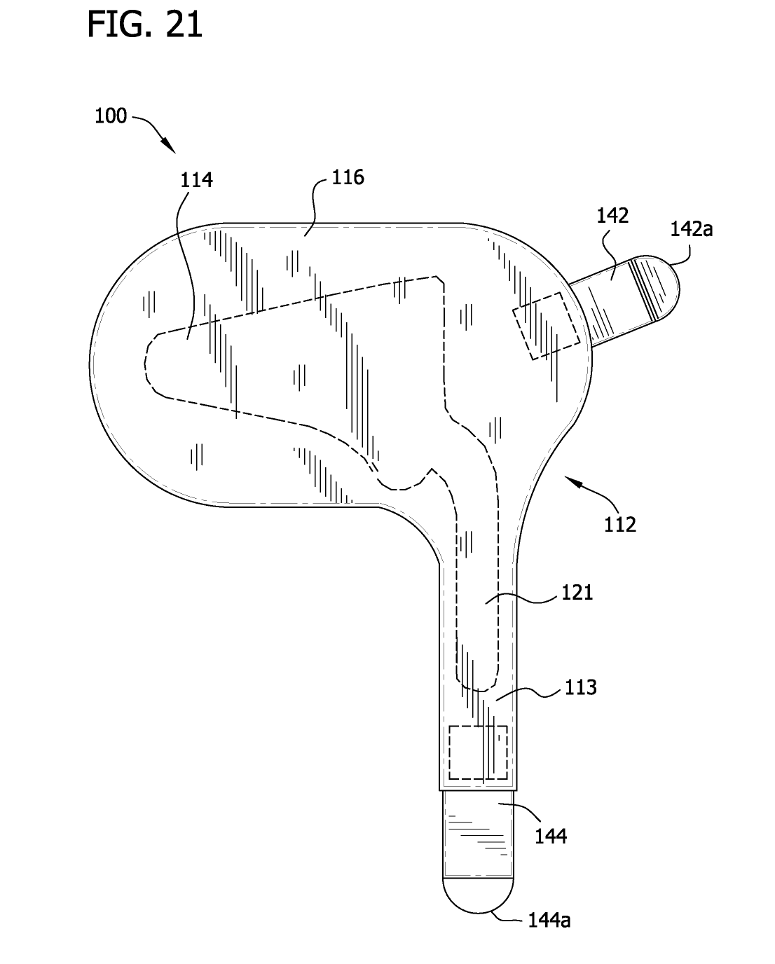

With reference to FIGS. 21-24, alternative embodiments of the compression apparatus 10 of FIGS. 1-4 are illustrated. These embodiments are similar to the embodiment illustrated in FIGS. 1-4 and will only be discussed in detail to the extent necessary to identify differences in construction and operation.

With particular reference to FIG. 21, compression apparatus 100 includes foot cuff 112 and inflatable member 114, shown in phantom. Foot cuff 112 includes first and second layers defining a space therebetween for receiving inflatable member 114 therein, which are similar to upper and lower inflatable layers 22, 24 (FIG. 1). Inflatable member 114 is configured for independent movement relative to at least one of first or second layers. Foot cuff 112 includes strap portion 113 extending longitudinally therethrough. A hook element 144a attached to a distal end of strap portion 113, and a hook element 142 is mounted to a portion of the foot cuff 112. Inflatable member 114 is configured and dimensioned for substantial fit within foot cuff 112, wherein foot cuff 112 is configured to be wrapped around the bottom portion of the foot. In FIG. 21, the heel portion 121 of the inflatable of the foot cuff wraps around the ankle of the foot.

With particular reference to FIG. 22, compression apparatus 200 includes foot cuff 212 and inflatable member 214, shown in phantom. Foot cuff 212 includes first and second layers defining a space therebetween for receiving inflatable member 214 therein, which are similar to upper and lower inflatable layers 22, 24 (FIG. 1). Inflatable member 214 is configured for independent movement relative to at least one of first or second layers. Inflatable member 214 includes a valve connector 216 for connecting inflatable member 214 to a pressurized fluid source. Valve connector 216 protrudes through an opening positioned on the outer cuff portion of foot cuff 212, in a manner described hereinabove with respect to compression apparatus 10. Foot cuff 212 includes strap portion 218 extending longitudinally therethrough. A hook element 220 attached to a distal end of strap portion 218, and a hook element 222 is mounted to a portion of the foot cuff 212. Inflatable member 214 is configured and dimensioned for substantial fit within foot cuff 212, wherein foot cuff 212 is configured to be wrapped around the bottom portion of the foot.

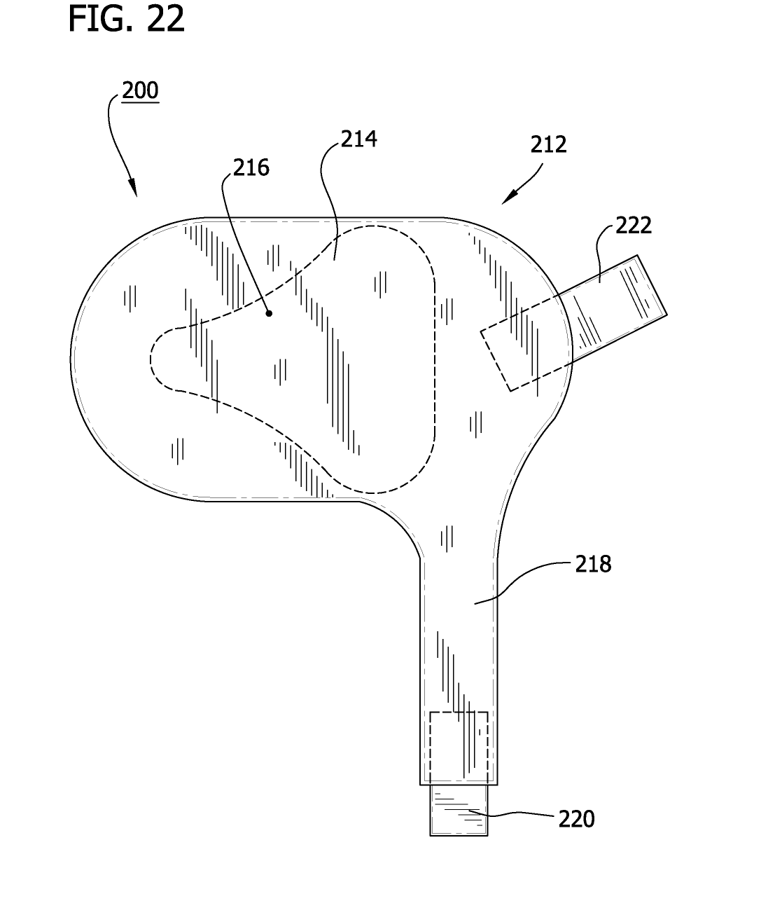

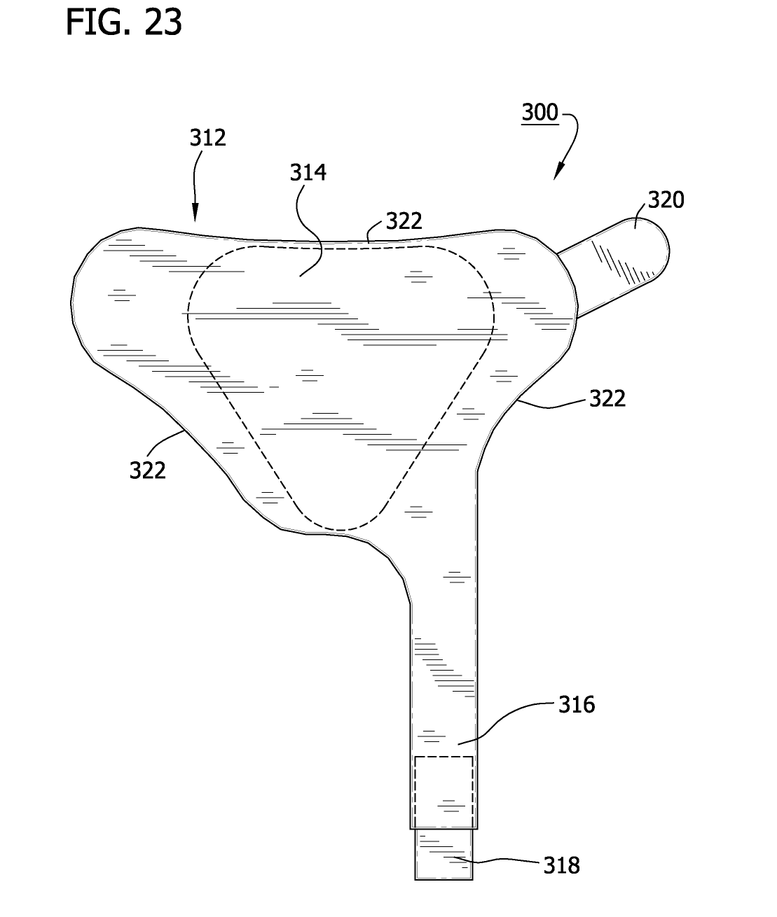

With reference to FIG. 23, compression apparatus 300 includes foot cuff 312 and inflatable member 314, shown in phantom. Foot cuff 312 includes first and second layers defining a space therebetween that receives inflatable member 314 therein, which are similar to upper and lower inflatable layers 22, 24 (FIG. 1). Inflatable member 314 is configured for independent movement relative to at least one of first or second layers. Foot cuff 312 further includes an elongated strap 316 extending longitudinally therethrough. Hook element 318 is mounted to elongated strap 316, while hook element 320 is mounted on foot cuff 312. Foot cuff 312 further includes a plurality of curvatures 322 for custom fitting about the foot.

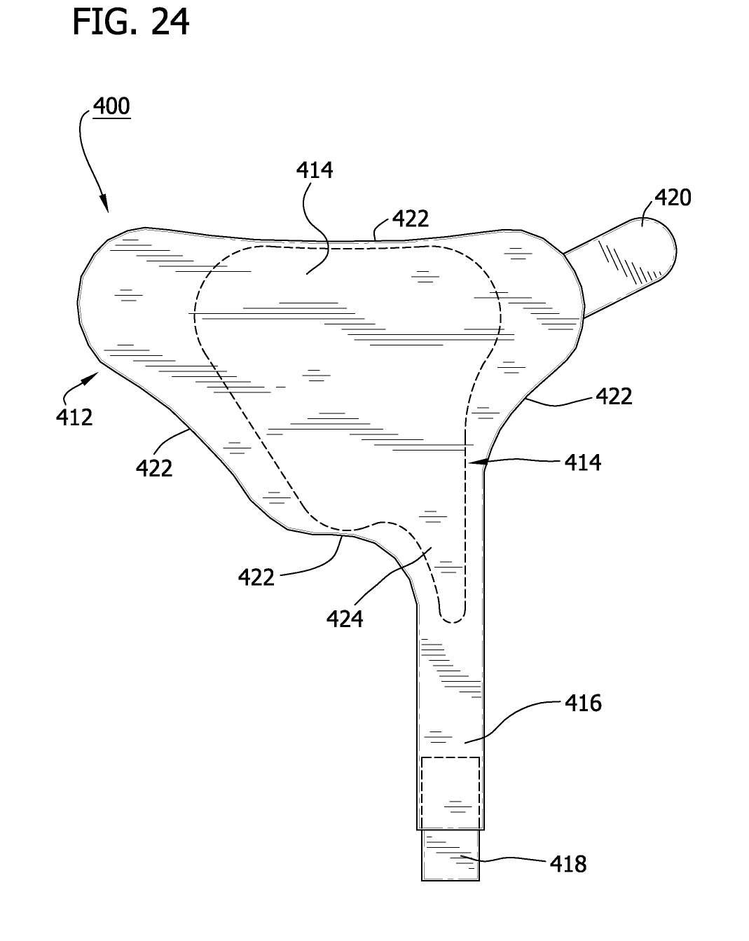

With reference to FIG. 24, compression apparatus 400 includes foot cuff 412 and inflatable member 414 shown in phantom. Foot cuff 412 includes first and second layers defining a space therebetween that receives inflatable member 414 therein. Inflatable member 414 is configured for independent movement relative to at least one of first or second layers. Foot cuff 412 includes an elongated strap 416 extending longitudinally therethrough. Hook element 418 is mounted to elongated strap 416, while hook element 420 is mounted on foot cuff 412. Foot cuff 412 further includes a plurality of curvatures 422 for custom fitting about the foot. Inflatable member 414 includes inflatable elongated strap portion 424 extending substantially along strap portion 416.

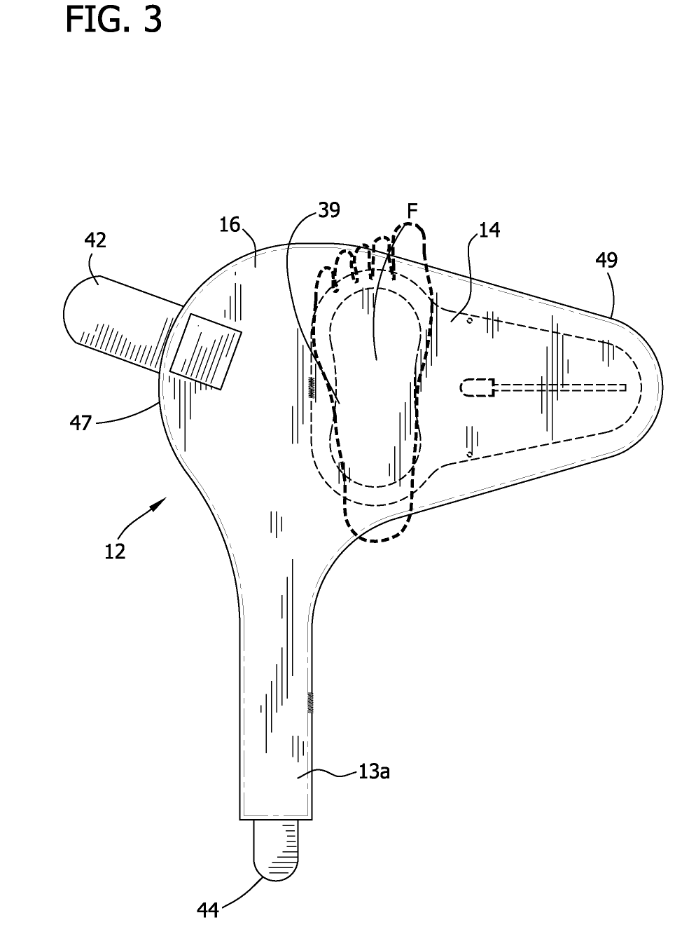

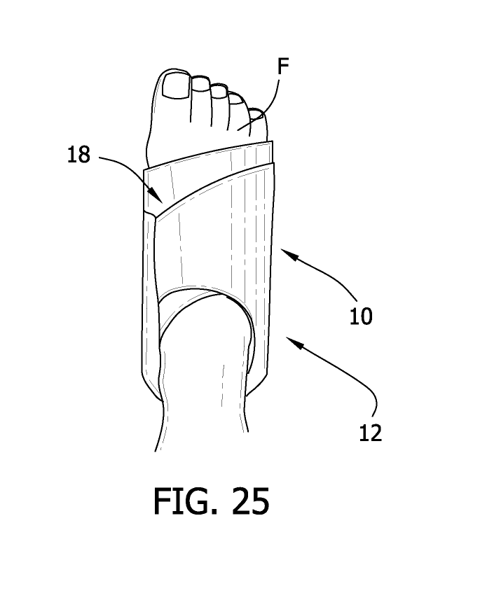

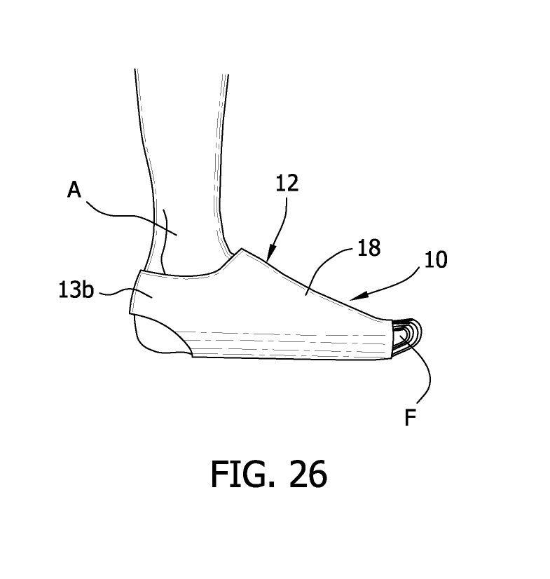

In use, compression apparatus 10, in accordance with the present disclosure, is configured to apply compressive forces to a patient's foot. Referring now to FIGS. 3 and 25-26, the cuff 12 is disposed about the foot F by wrapping the first flap 49 about the medial aspect of the foot F over the instep. The second flap 47 is then wrapped over the first flap 49 and instep and secures to the outer layer 18 by hook fastener 42. The strap portions 13a,13b are then wrapped around the ankle A (FIG. 26) and the hook fastener 44 engages the outer surface of the outer layer 18. After placement of foot cuff 12 about foot F and connecting movable inflatable member 14 to pressurized fluid source 38 via inflation assembly 32, controller 40 may then be actuated for supply pressurized air to compression apparatus 10 and initiating compression therapy. Controller 40 intermittently inflates inflatable member 14 during periodic compression cycles in a pressure gradient profile. As compression therapy is applied, contact layer 16 and inflatable member 14 move independently, while outer layer 18 remains fixed against the foot throughout the compression therapy. The wicking properties of contact layer 16 will facilitate keeping feet F dry during prolonged periods of compression therapy. Deflation between successive inflation cycles occurs by return of air through inflatable member 14 to controller 40, as known in the art

With reference to FIGS. 27-28, another embodiment of a compression apparatus in accordance with the present disclosure is illustrated and is designated generally as compression apparatus 500. Compression apparatus 500 is adapted for use in a system for applying compressive pressure to a portion of a patient's body, such as, for example, the legs. Compression apparatus 500 is similar to the compression cuff disclosed in U.S. Pat. No. 5,626,556 to Tobler et al. and U.S. Pat. No. 5,795,312 to Dye that are currently owned by Tyco Healthcare Group LP and are incorporated herein by reference in their entirety.

With particular reference to FIG. 27, the compression apparatus 500, in accordance with the present disclosure, includes cuff 510 having first or outer sheet 512 and second or inner sheet 514 connected by a plurality of laterally extending sealing lines 516 and longitudinally extending sealing lines 518 connecting the ends of lateral sealing lines 516. Outer sheet 512 is adapted as an outer gas-impervious sheet and second sheet 514 is adapted as an inner gas-impervious sheet, for placement against the person's limbs. Sealing lines 516, 518 may be formed by radio frequency (RF) welding, etc. An elongated opening 521 is provided for extending through what would be the knee region. Opening 521 is defined by peripheral edges 523 extending around opening 521.

Sealing lines 516, 518 define a plurality of spaces or chambers 520a, 520b, and 520c that are adapted for receiving movable inflatable members 522a, 522b and 522c. Inflatable members 522a, 522b, and 522c are configured for moving independently relative to cuff 510. Similar to inflatable member 14 of compressive cuff 10, inflatable members 522a, 522b and 522c are adapted for receiving and retaining a pressurized fluid, such as, for example, air, for exerting compressive pressure to the leg of the patient during successive pressure applying cycles. A plurality of lumens 524a, 524b, 524c, and 524d having a valve connector 525 is included for operably connecting inflatable members 522a, 522b and 522cto a controller (not shown) having a source of pressurized fluid, such as, air.

First or outer sheet 512 may, for example, comprise a suitable flexible polymeric material, such as, for example, polyvinyl chloride (PVC) on the order of 5-10 mils thick. Second or inner sheet 514 will preferably comprise a similar polymeric material, e.g. 5-10 mil PVC having laminated to the inner surface to be placed against the limb a non-women material such as polyester for added comfort to the wearer.

Compression apparatus 500 further includes a plurality of hook fasteners for attaching the cuff about the patient's limb. Hook fasteners include a set of spaced strips, such as loop material, positioned on first or outer sheet 512 and cooperating with a set of spaced hook material 526a, 526b, and 526c disposed on second or inner sheet 514 for releasably fastening compression apparatus 500 encircling the limb.

With particular reference to FIG. 27, in use, after placement of cuff 510 on the patient's leg and connection to the controller via connector 525 and plurality of lumens 524a, 524b, 524c and 524d, the controller intermittently inflates inflatable members 522a, 522b and 522c sequentially during periodic compression cycles in a pressure gradient profile. As compression therapy is applied, first or outer sheet 512 and inflatable members 522a, 522b and 522c move independently, while second or inner sheet 514 remains fixed against the leg throughout the compression therapy. Deflation between successive inflation cycles occurs by return of air through inflatable members 522a, 522b, and 522c to the controller, as known in the art.

It will be understood that numerous modifications and changes in form and detail may be made to the embodiments of the present disclosure. It is contemplated that numerous other configuration of the compression apparatus and geometries and orientation of the inflatable member may be used, and the material of the cuff and/or inflatable member may be selected from numerous materials other than those specifically disclosed. Therefore, the above description should not be construed as limiting the disclosed compression apparatus but merely as exemplifications of embodiments thereof. Those skilled in the art will envision numerous modifications within the scope of the present disclosure as defined by the claims appended hereto.

What is claimed is:

1. A compression apparatus comprising:
a first layer and a second layer defining a space therebetween; and
only a single bladder within an inflatable member disposed in the space, the inflatable member being freely movable in relation to at least one of the first and second layers when disposed on the body part of a patient for providing DVT prophylaxis therapy to a limb of a patient.

2. A compression apparatus as set forth in claim 1, wherein an outer surface of the inflatable member is secured to an inner surface of the first or second layer by an adhesive layer selected from the group consisting of:
at least one strip of adhesive tape;
an adhesive fluid;
a gel; and
combinations thereof.

3. A compression apparatus as set forth in claim 2 wherein the adhesive layer comprises plural, spaced apart strips of double-sided adhesive tape joining the bladder to the first or second layer.

4. A compression apparatus as set forth in claim 2 wherein the adhesive fluid is selected from the group consisting of glue, a thermoplastic adhesive, a thermosetting adhesive, a rubber-resin blend, ultraviolet curable adhesives and mixtures thereof.

5. A compression apparatus as set forth in claim 2 wherein the gel comprises silicone gels, hydrocolloidal gels, cyanoacrylate gels, thixotropic gels and mixtures thereof.

6. A compression apparatus as set forth in claim 2 wherein the inflatable member has a body portion and a strap portion, the body portion adapted to engage a foot and the strap portion adapted to engage an ankle.

7. A compression apparatus as set forth in claim 2 wherein the inflatable member has a body portion that is adapted to engage a foot.

8. A compression apparatus as set forth in claim 2 further comprising a rigid sole element.
